(19) United States
(12) Patent Application Publication
(10) Pub. No.: US 2006/0078896 A1
(43) Pub. Date: Apr. 13, 2006

(54) Method, system, and computer program product to assess properties of a chemical array

(76) Inventors: Bill J. Peck, Mountain View, CA (US); Eric M. Leproust, San Jose, CA (US)

Correspondence Address: AGILENT TECHNOLOGIES, INC.;INTELLECTUAL PROPERTY ADMINISTRATION, LEGAL DEPT., P.O. BOX 7599, M/S DL429, LOVELAND, CO, 80537-0599, US

(21) Appl. No.: 10/964,063

(22) Filed: Oct. 12, 2004

(51) Int. Cl. C12Q 1/68; G01N 33/53; G06F 19/00; C40B 40/08

(52) U.S. Cl. 435/6; 435/7.1; 702/19

(57) ABSTRACT

A method, a system, and a computer-readable medium storing a program for determining array quality during in-situ manufacturing of an array includes determining an average feature coverage fraction of the array, determining a background area fraction of the array, measuring a contact angle of the background, and determining the array quality based upon the measured, feature contact angle.

### BACKGROUND

Chemical arrays, such as polynucleotide or protein arrays (for example, DNA or RNA arrays) may be used as diagnostic or screening tools. Polynucleotide arrays include regions of usually different sequence polynucleotides arranged in a predetermined configuration on a substrate. These regions (sometimes referred to as “features”) are positioned at respective locations (“addresses”) on the substrate. The arrays, when exposed to a sample, will exhibit an observed binding pattern. This binding pattern can be detected upon reading the array. For example all polynucleotide targets (for example, DNA) in the sample can be labeled with a suitable label (such as a fluorescent compound), and the fluorescence pattern on the array accurately observed following exposure to the sample. Assuming that the different sequence polynucleotides were correctly deposited in accordance with the predetermined configuration, then the observed binding pattern will be indicative of the presence and/or concentration of one or more polynucleotide components of the sample.

Biopolymer arrays can be fabricated by depositing previously obtained biopolymers (such as from synthesis or natural sources) onto a substrate, or by in situ synthesis methods.

Methods of depositing obtained biopolymers include loading then touching a pin or capillary to a surface, such as described in U.S. Pat. No. 5,807,522 or deposition by firing from a pulse jet such as an inkjet head, such as described in PCT publications WO 95/25116 and WO 98/41531, and elsewhere. Such a deposition method can be regarded as forming each feature by one cycle of attachment (that is, there is only one cycle at each feature during which the previously obtained biopolymer is attached to the substrate). For in situ fabrication methods, multiple different reagent droplets are deposited by pulse jet or other means at a given target location in order to form the final feature (hence a probe of the feature is synthesized on the array substrate). The in situ fabrication methods include those described in U.S. Pat. No. 5,449,754 for synthesizing peptide arrays, and in U.S. Pat. No. 6,180,351 and WO 98/41531 and the references cited therein for polynucleotides, and may also use pulse jets for depositing reagents.

The in situ method for fabricating a polynucleotide array typically follows, at each of the multiple different addresses at which features are to be formed, the same conventional iterative sequence used in forming polynucleotides from nucleoside reagents on a support by means of known chemistry. This iterative sequence can be considered as multiple ones of the following attachment cycle at each feature to be formed: (a) coupling an activated selected nucleoside (a monomeric unit) through a phosphite linkage to a functionalized support in the first iteration, or a nucleoside bound to the substrate (i.e. the nucleoside-modified substrate) in subsequent iterations; (b) optionally, blocking unreacted hydroxyl groups on the substrate bound nucleoside (sometimes referenced as “capping”); (c) oxidizing the phosphite linkage of step (a) to form a phosphate linkage; and (d) removing the protecting group (“deprotection”) from the now substrate bound nucleoside coupled in step (a), to generate a reactive site for the next cycle of these steps. The coupling can be performed by depositing drops of an activator and phosphoramidite at the specific desired feature locations for the array. A final deprotection step is provided in which nitrogenous bases and phosphate group are simultaneously deprotected by treatment with ammonium hydroxide and/or methylamine under known conditions. Capping, oxidation and deprotection can be accomplished by treating the entire substrate (“flooding”) with a layer of the appropriate reagent.

The functionalized support (in the first cycle) or deprotected coupled nucleoside (in subsequent cycles) provides a substrate bound moiety with a linking group for forming the phosphite linkage with a next nucleoside to be coupled in step (a). Final deprotection of nucleoside bases can be accomplished using alkaline conditions such as ammonium hydroxide, in another flooding procedure in a known manner. Conventionally, a single pulse jet or other dispenser is assigned to deposit a single monomeric unit.

The foregoing chemistry of the synthesis of polynucleotides is described in detail, for example, in Caruthers, Science 230: 281-285, 1985; Itakura et al., Ann. Rev. Biochem. 53: 323-356; Hunkapillar et al., Nature 310: 105-110, 1984; and in “Synthesis of Oligonucleotide Derivatives in Design and Targeted Reaction of Oligonucleotide Derivatives”, CRC Press, Boca Raton, Fla., pages 100 et seq., U.S. Pat. No. 4,458,066, U.S. Pat. No. 4,500,707, U.S. Pat. No. 5,153,319, U.S. Pat. No. 5,869,643, EP 0294196, and elsewhere. The phosphoramidite and phosphite triester approaches are most broadly used, but other approaches include the phosphodiester approach, the phosphotriester approach and the H-phosphonate approach. The substrates are typically functionalized to bond to the first deposited monomer. Suitable techniques for functionalizing substrates with such linking moieties are described, for example, in U.S. Pat. No. 6,258,454 and Southern, E. M., Maskos, U. and Elder, J. K., Genomics, 13, 1007-1017, 1992. In the case of array fabrication, different monomers and activator may be deposited at different addresses on the substrate during any one cycle so that the different features of the completed array will have different desired biopolymer sequences. One or more intermediate further steps may be required in each cycle, such as the conventional oxidation, capping and washing steps in the case of in situ fabrication of polynucleotide arrays (again, these steps may be performed in flooding procedure).

Further details of fabricating biopolymer arrays by depositing either previously obtained biopolymers or by the in situ method are disclosed in U.S. Pat. No. 6,242,266, U.S. Pat. No. 6,232,072, U.S. Pat. No. 6,180,351, and U.S. Pat. No. 6,171,797. Particularly useful linker compositions and methods are disclosed in U.S. Pat. Nos. 6,319,674 and 6,444,268. These patents also provide a means by which the surface energy of a substrate can be modified to control deposited drop spread during array fabrication.

In array fabrication, the quantities of polynucleotide available are usually very small and expensive. Additionally, sample quantities available for testing are usually also very small. These conditions require use of arrays with small, closely spaced features. For example, arrays may have several thousand features present. However, in some situations arrays with far fewer features (for example, only one hundred features) are sufficient. Multiple arrays with fewer features can be accommodated on a same substrate and exposed to different samples.

The quality of DNA arrays is typically assessed after manufacture, i.e., after probes are immobilized on the array and after the complete synthesis of the DNA array and after completion of any post processing steps. That is, DNA arrays are not presently subjected to a quality control inspection until after they are hybridized, which is a time-consuming and expensive task. During the actual printing of the DNA array, very little is known about the quality of the molecules being placed on the DNA array. Although surface energy measurements may provide useful information about array quality, it is difficult to measure the surface energy on a feature-to-feature basis.

It is desirable to assess the quality of the DNA arrays during the manufacturing process since identification of DNA arrays suffering from low-quality synthesis early in the manufacturing process would decrease time wasted in completing further manufacturing of the low-quality DNA array, including time during which the manufacturing machine is occupied by the low-quality DNA array while successive layers are printed and time required for the subsequent post processing steps.

### SUMMARY OF THE INVENTION

In one aspect, the present invention provides a method in which a property of a chemical array, such as a nucleic acid array, is assessed using a contact angle measurement taken over a region including at least a portion of a feature on the array. In one aspect, the method comprises measuring contact angle of liquid (e.g., such as water) over a region including one or more features. In another aspect, the method comprises measuring a contact angle of a liquid over an array or a portion of an array on a substrate comprising multiple arrays. The measured array, in certain aspects may be smaller (e.g., in terms of numbers of features) than other arrays on the substrate. Contact angle measurement can be used to evaluate in situ synthesis of probes on an array. In one aspect, this evaluation may be used to make a decision as to whether to continue synthesis of the array.

It is another aspect of the present invention to provide an ability to assess how much DNA mass is being grown in features too small to be measured using a standard contact angle measurement. In one aspect, an identifier is assigned to the array that provides information or is associated with information relating to an amount of DNA mass present at one or more features of the array. The above aspects can be attained by a method and a system that determines array quality during manufacturing of an array. The method and system of the present invention determines an average feature coverage fraction of the array, determines a background area fraction of the array, measures a contact angle of the background, and determines the array quality based upon the measured, feature contact angle. These together with other aspects and advantages which will be subsequently apparent, reside in the details of construction and operation as more fully hereinafter described and claimed, reference being had to the accompanying drawings forming a part hereof, wherein like numerals refer to like parts throughout.

### BRIEF DESCRIPTION OF THE DRAWINGS

FIG. 1 shows a relationship between an array, contact angles and a surface.

FIG. 2 shows a measured contact angle related to the present invention.

FIG. 3 shows a flowchart illustrating a method according to one aspect of the invention

FIG. 4 is a block diagram illustrating a system according to one aspect of the invention.

### DETAILED DESCRIPTION

Reference will now be made in detail to the embodiments of the present invention, examples of which are illustrated in the accompanying drawings, wherein like reference numerals refer to the like elements throughout. The embodiments are described below in order to explain the present invention by referring to the figures.

In the present application, unless a contrary intention appears, the following terms refer to the indicated characteristics.

It must be noted that as used herein and in the appended claims, the singular forms “a”, “and”, and “the” include plural referents unless the context clearly dictates otherwise.

A “biopolymer” is a polymer of one or more types of repeating units. Biopolymers are typically found in biological systems and particularly include polysaccharides (such as carbohydrates), and peptides (which term is used to include polypeptides, and proteins whether or not attached to a polysaccharide) and polynucleotides as well as their analogs such as those compounds composed of or containing amino acid analogs or non-amino acid groups, or nucleotide analogs or non-nucleotide groups. This includes polynucleotides in which the conventional backbone has been replaced with a non-naturally occurring or synthetic backbone, and nucleic acids (or synthetic or naturally occurring analogs) in which one or more of the conventional bases has been replaced with a group (natural or synthetic) capable of participating in Watson-Crick type hydrogen bonding interactions. Polynucleotides include single or multiple stranded configurations, where one or more of the strands may or may not be completely aligned with another.

A “nucleotide” refers to a sub-unit of a nucleic acid and has a phosphate group, a 5 carbon sugar and a nitrogen containing base, as well as functional analogs (whether synthetic or naturally occurring) of such sub-units which in the polymer form (as a polynucleotide) can hybridize with naturally occurring polynucleotides in a sequence specific manner analogous to that of two naturally occurring polynucleotides. For example, a “biopolymer” includes DNA (including cDNA), RNA, oligonucleotides, and PNA and other polynucleotides as described in U.S. Pat. No. 5,948,902 and references cited therein (all of which are incorporated herein by reference), regardless of the source.

An “oligonucleotide” generally refers to a nucleotide multimer of about 10 to 100 nucleotides in length, while a “polynucleotide” includes a nucleotide multimer having any number of nucleotides.

A “biomonomer” references a single unit, which can be linked with the same or other biomonomers to form a biopolymer (for example, a single amino acid or nucleotide with two linking groups one or both of which may have removable protecting groups).

A “biopolymer precursor” are smaller units of a biopolymer which may be chemically bonded end to end to form the biopolymers. Biomonomers are one type of biopolymers precursors, but biopolymer precursors could include two or more linked monomer units. A fluid references a liquid (for example, a solution of biopolymer or biopolymer precursor).

As used herein an “array,” includes any two-dimensional or substantially two-dimensional (as well as a three-dimensional) arrangement of addressable regions bearing a particular chemical moiety or moieties (e.g., biopolymers such as polynucleotide or oligonucleotide sequences (nucleic acids), polypeptides (e.g., proteins), carbohydrates, lipids, etc.) associated with that region. In the broadest sense, arrays are arrays of polymeric binding agents, where the polymeric binding agents may be any of: polypeptides, proteins, nucleic acids, polysaccharides, synthetic mimetics of such biopolymeric binding agents, etc. In many embodiments of interest, the arrays are arrays of nucleic acids, including oligonucleotides, polynucleotides, cDNAs, mRNAs, synthetic mimetics thereof, and the like. Where the arrays are arrays of nucleic acids,the nucleic acids may be covalently attached to the arrays at any point along the nucleic acid chain, but are generally attached at one of their termini (e.g. the 3′ or 5′ terminus). Sometimes, the arrays are arrays of polypeptides, e.g., proteins or fragments thereof.

Any given substrate may carry one, two, four or more or more arrays disposed on a front surface of the substrate. Depending upon the use, any or all of the arrays may be the same or different from one another and each may contain multiple spots or features. A typical array may contain more than ten, more than one hundred, more than one thousand more ten thousand features, or even more than one hundred thousand features, in an area of less than 20 cm² or even less than 10 cm². For example, features may have widths (that is, diameter, for a round spot) in the range from about 10 μm to 1.0 cm. In other embodiments each feature may have a width in the range of about 1.0 μm to 1.0 mm, usually about 5.0 μm to 500 μm, and more usually 10 μm to 200 μm. Non-round features may have area ranges equivalent to that of circular features with the foregoing width (diameter) ranges. At least some, or all, of the features are of different compositions (for example, when any repeats of each feature composition are excluded the remaining features may account for at least 5%, 10%, or 20% of the total number of features).

All of the features may be different, or some could be the same (for example, when any repeats of each feature composition are excluded the remaining features may account for at least 5%, 10%, or 20% of the total number of features). In some aspects, features are arranged in straight line rows extending left to right. In the case where arrays are formed by the in situ or deposition of previously obtained biopolymers by depositing for each feature a droplet of reagent in each cycle such as by using a pulse jet such as an inkjet type head, interfeature areas will typically be present which do not carry any polynucleotide or moieties of the array features. It will be appreciated though, that the interfeature areas could be of various sizes and configurations. It will also be appreciated that there need not be any space separating arrays in a multi-array substrate from one another although there typically will be. As per usual, A, C, G, T represent the usual nucleotides. It will be understood that there may be a linker molecule (not shown) of any known types between the front surface and the first nucleotide.

Interfeature areas will typically (but not essentially) be present which do not carry any polynucleotide (or other biopolymer or chemical moiety of a type of which the features are composed). Such interfeature areas typically will be present where the arrays are formed by processes involving drop deposition of reagents but may not be present when, for example, photolithographic array fabrication processes are used. It will be appreciated though, that the interfeature areas, when present, could be of various sizes and configurations.

Each array may cover an area of less than 100 cm², or even less than 50 cm², 10 cm² or 1 cm². In many embodiments, the substrate carrying the one or more arrays will be shaped generally as a rectangular solid (although other shapes are possible), having a length of more than 4 mm and less than 1 m, usually more than 4 mm and less than 600 mm, more usually less than 400 mm; a width of more than 4 mm and less than 1 m, usually less than 500 mm and more usually less than 400 mm; and a thickness of more than 0.01 mm and less than 5.0 mm, usually more than 0.1 mm and less than 2 mm and more usually more than 0.2 and less than 1 mm. With arrays that are read by detecting fluorescence, the substrate may be of a material that emits low fluorescence upon illumination with the excitation light. Additionally in this situation, the substrate may be relatively transparent to reduce the absorption of the incident illuminating laser light and subsequent heating if the focused laser beam travels too slowly over a region. For example, a substrate may transmit at least 20%, or 50% (or even at least 70%, 90%, or 95%), of the illuminating light incident on the front as may be measured across the entire integrated spectrum of such illuminating light or alternatively at 532 nm or 633 nm.

An array is “addressable” when it has multiple regions of different moieties (e.g., different polynucleotide sequences) such that a region (i.e., a “feature” or “spot” of the array) at a particular predetermined location (i.e., an “address”) on the array will detect a particular target or class of targets (although a feature may incidentally detect non-targets of that feature). Array features are typically, but need not be, separated by intervening spaces.

In the case of an array, the “target” will be referenced as a moiety in a mobile phase (typically fluid), to be detected by probes (“target probes”) which are bound to the substrate at the various regions. However, either of the “target” or “target probes” may be the one which is to be evaluated by the other (thus, either one could be an unknown mixture of polynucleotides to be evaluated by binding with the other).

An “array layout” or “array characteristics”, are used interchangeably to refer to one or more physical, chemical or biological characteristics of the array, such as feature positioning within an array and array positioning on a substrate, one or more feature dimensions, or some indication of an identity or function (for example, chemical or biological) of a moiety at a given feature location, or how the array should be handled (for example, conditions under which the array is exposed to a sample, or array reading specifications or expected signals or signal ranges from control features on the array following sample exposure).

“Hybridizing” and “binding”, with respect to polynucleotides, are used interchangeably.

“Control features” on the array are those features which are present to provide a check on array or sample performance during use of the array. For example control features may be negative controls (very little or no signal expected after sample exposure) or positive controls (high signal expected after sample exposure).

A “plastic” is any synthetic organic polymer of high molecular weight (for example at least 1,000 grams/mole, or even at least 10,000 or 100,000 grams/mole.

“Flexible” with reference to a substrate or substrate web, references that the substrate can be bent 180 degrees around a roller of less than 1.25 cm in radius. The substrate can be so bent and straightened repeatedly in either direction at least 100 times without failure (for example, cracking) or plastic deformation. This bending must be within the elastic limits of the material. The foregoing test for flexibility is performed at a temperature of 20° C.

A “web” references a long continuous piece of substrate material having a length greater than a width. For example, the web length to width ratio may be at least 5/1, 10/1, 50/1, 100/1, 200/1, or 500/1, or even at least 1000/1.

A “remote location,” refers to location other than the location at which the array is present and hybridization occurs. For example, a remote location could be another location (e.g., office, lab, etc.) in the same city, another location in a different city, another location in a different state, another location in a different country, etc. As such, when one item is indicated as being “remote” from another, what is meant is that the two items are at least in different rooms or different buildings, and may be at least one mile, ten miles, or at least one hundred miles apart.

“Communicating information” refers to transmitting the data representing that information as signals (e.g., electrical, optical, radio, magnetic, etc) over a suitable communication channel (e.g., a private or public network).

“Forwarding” an item refers to any means of getting that item from one location to the next, whether by physically transporting that item or otherwise (where that is possible) and includes, at least in the case of data, physically transporting a medium carrying the data or communicating the data. An array “assembly” may be the array plus only a substrate on which the array is deposited, although the assembly may be in the form of a package which includes other features (such as a housing with a chamber).

It will also be appreciated that throughout the present application, that words such as “front”, “back”, “top”, “upper”, and “lower” are used in a relative sense only. Reference to a singular item, includes the possibility that there are plural of the same items present.

“May” refers to optionally. Any recited method can be carried out in the ordered sequence of events as recited, or any other logically possible sequence.

A “pulse jet” is any device which can dispense drops in the formation of an array. Pulse jets operate by delivering a pulse of pressure (such as by a piezoelectric or thermoelectric element) to liquid adjacent an outlet or orifice such that a drop will be dispensed therefrom.

A “linking layer” bound to the surface may, for example, be less than 200 angstroms or even less than 10 angstroms in thickness (or less than 8, 6, or 4 angstroms thick). Such layer may have a polynucleotide, protein, nucleoside or amino acid minimum binding affinity of 10⁴ to 10⁶ units/μ². Layer thickness can be evaluated using UV or X-ray elipsometry if desired.

“Physically uninterrupted” in reference to the substrate surface means there are no physical barriers present which can contain liquid to prevent it from spreading beyond that array. Physical barriers are barriers of sufficiently large dimensions to prevent fluid flow between arrays and are distinguished from liquid containment barriers resulting from discontinuities in surface energy due to chemical composition (for example, the interface at a hydrophobic region and a less hydrophobic adjacent region). For example, a physically uninterrupted surface may be one which has no physical barriers such as walls surrounding the arrays which extend above the substrate surface more than 10 micrometers (or more than 5, 2, or 1 micrometers).

A “region” on the surface is a contiguous surface portion, that is connected. A region may have discontinuities within the region (for example, an inter-array region is interconnected but within it there are regions carrying the features which regions are not part of the inter-array region).

A “continuous” region is one which is uninterrupted (that is, it extends completely between its outer dimensions). Thus, the continuous region carrying the features is that continuous portion of the substrate carrying the features and everything between them (and thus also includes the inter-feature region). When the continuous region carrying the features is referenced as being physically uninterrupted, then this refers to their being no physical barriers between the outer boundaries of the surface portion within which the arrays lie.

“Surface energy” (typically measured in ergs/cm²) of a liquid or solid substance pertains to the free energy of a molecule on the surface of the substance, which is necessarily higher than the free energy of a molecule contained in the in the interior of the substance; surface molecules have an energy roughly 25% above that of interior molecules. The term “surface tension” refers to the tensile force tending to draw surface molecules together, and although measured in different units (as the rate of increase of surface energy with area, in dynes/cm), is numerically equivalent to the corresponding surface energy.

“Contact angle” of a liquid with a surface is the acute angle measured between the edge of a drop of liquid on that surface and the surface. Contact angle measurements are well known and can be obtained by various instruments such as an FTA200 available from First Ten Angstroms, Portsmouth, Va., U.S.A. Surfaces which are more hydrophobic (which have a higher surface energy) will have higher contact angles with water or aqueous liquids than surfaces which are less hydrophobic (for example, a hydrophobic surface may have a water drop contact angle of more than 50 degrees, or even more than 90 degrees). The contact angle of an array (sometimes referenced as the “average contact angle” or “effective contact angle”) is the average contact angle of the features of that array and the inter-feature areas. Contact angles are measured with water unless otherwise indicated.

“Linker agent density” or “capping agent density” refers to the number of linker molecules or capping molecules per unit area. Linker agents are counted in determining linker agent density whether or not they are linked to probes or are themselves capped. For capping agent density only capping agents directly attached to the substrate surface are counted in the capping agent density. If different regions on a substrate surface of uniform composition are exposed under the same conditions to a same composition of linking agent which binds to the surface at a same density, the linker agent density in the regions will be considered to be the “same”.

“Probe density” is a shorthand way of referring to the number of linker molecules or probe molecules per unit area within a feature. This term then is used interchangeably with, and has the same meaning as “feature probe density”. Thus, any inter-feature areas which are essentially devoid of the probe are not taken into consideration in determining a probe density. “Probe density” in a region then, is distinct and independent of feature density (which is the number of features per unit area).

The general features of an array are now described. Additional features of arrays may be found in, for example, U.S. patent publication 20040152083.

In one aspect, an array assembly includes a substrate comprising a plurality of addressable features arrayed on the surface of the substrate.

The substrate may be formed of a variety of materials and the size and shape of the substrate is not a limiting feature of the invention. The substrate may be rigid or flexible or semi-flexible. The term “rigid” is used herein to refer to a structure e.g., a bottom surface that does not readily bend without breakage, i.e., the structure is not flexible. The term “flexible” is used herein to refer to a structure, e.g., a bottom surface or a cover, that is capable of being bent, folded or similarly manipulated without breakage. For example, a cover is flexible if it is capable of being peeled away from the bottom surface without breakage. In one aspect, the substrate comprises a flexible web that can be bent 180 degrees around a roller of less than 1.25 cm in radius at a temperature of 20° C. As used herein, a “web” refers to a long continuous piece of substrate material having a length greater than a width. For example, the web length to width ratio may be at least 5/1, 10/1, 50/1, 100/1, 200/1, or 500/1, or even at least 1000/1. A web substrate may be of various lengths including at least about 1 m, at least about 2 m, or at least about 5 m (or even at least about 10 m).

Rigid solid supports may be made from silicon, glass, rigid plastics, e.g. polytetrafluoroethylene, polypropylene, polystyrene, polycarbonate, etc., or metals, e.g. gold, platinum, etc. Flexible solid supports may be made from a variety of materials, such as, for example, nylon, nitrocellulose, polypropylene, polyester films, e.g., polyethylene terephthalate, polymethyl methacrylate or other acrylics, polyvinyl chloride or other vinyl resin. Various plasticizers and modifiers may be used with polymeric substrate materials to achieve selected flexibility characteristics.

Solid supports may exist in a variety of configurations ranging from simple to complex. Suitable substrates may exist, for example, as gels, sheets, tubing, spheres, containers, pads, slices, films, plates, slides, strips, plates, disks, rods, particles, beads, etc. The substrate is preferably flat, but may take on alternative surface configurations. The substrate can be a flat glass substrate, such as a conventional microscope glass slide, a cover slip and the like. Common substrates used for the arrays of probes are surface-derivatized glass or silica, or polymer membrane surfaces, as described in Guo, Z. et al. (cited above) and Maskos, U. et al., Nucleic Acids Res, 1992, 20:1679-84 and Southern, E. M. et al., Nucleic acids Res, 1994, 22:1368-73.

With arrays that are read by detecting fluorescence, the substrate may be of a material that emits low fluorescence upon illumination with the excitation light. Additionally in this situation, the substrate may be relatively transparent to reduce the absorption of the incident illuminating laser light and subsequent heating if the focused laser beam travels too slowly over a region. For example, substrate 10 may transmit at least 20%, or 50% (or even at least 70%, 90%, or 95%), of the illuminating light incident on the front as may be measured across the entire integrated spectrum of such illuminating light or alternatively at 532 nm or 633 nm.

An array assembly may comprise one or more arrays.

An array can be designed for testing against any type of sample, whether: a trial sample; reference sample; a combination of the foregoing; or a known mixture of polynucleotides, proteins, polysaccharides and the like (in which case the arrays may be composed of features carrying unknown sequences to be evaluated). In an array assembly comprising multiple arrays, depending upon intended use, any or all of arrays may be the same or different from one another. In one aspect, each array contains multiple spots or features of biopolymers in the form of polynucleotides.

A typical array 12 may contain from more than five, ten, twenty, thirty, one hundred, or even at least one hundred and fifty features. For example, features may have widths (that is, diameter, for a round spot) in the range from a 10 μm to 1.0 cm. In other embodiments each feature may have a width in the range of 1.0 μm to 1.0 mm, usually 5.0 μm to 500 μm, and more usually 10 μm to 200 μm. Non-round features may have area ranges equivalent to that of circular features with the foregoing width (diameter) ranges. At least some, or all, of the features are of different compositions (for example, when any repeats of each feature of the same composition are excluded, the remaining features may account for at least 5%, 10%, or 20% of the total number of features).

In the case where arrays are formed by the conventional in situ or deposition of previously obtained moieties, as described above, by depositing for each feature a droplet of reagent in each cycle such as by using a pulse jet such as an inkjet type head, inter-feature areas will typically be present which do not carry any polynucleotide. It will be appreciated though, that the inter-feature areas could be of various sizes and configurations. Each feature carries a predetermined polynucleotide (which includes the possibility of mixtures of polynucleotides). As per usual, A, C, G. T represent the usual nucleotides. “Link” represents a linking agent (molecule) covalently bound to the front surface and a first nucleotide, and “Cap” represents a molecule which does not bind to a nucleotide, as further described below.

Substrates may also include one or more identifiers in the form of bar codes. Identifiers such as other optical or magnetic identifiers or data storage elements could be used instead of bar codes which will carry information. Each identifier may be associated with each array on a substrate by being on the same substrate and therefore having a fixed location in relation to bar code from which relative location the identity of each array can be determined. The substrate may further have one or more fiducial marks for alignment purposes during array fabrication and reading. In one aspect, the identifier provides a means to identify information about probes defining features of each array, for example, such as sequence information. Alternatively, or additionally, an identifier may provide information (e.g., by being linked to such information in a relational database) relating to polymer mass (such as nucleic acid mass) on an array with which the identifier is associated.

An array can be fabricated by first functionalizing all of a substrate surface with the silanes in the manner described in U.S. Pat. No. 6,444,268. The arrays can then be fabricated on surface by forming the features using the in situ or deposition of previously obtained biopolymer fabrication methods described above or other methods known in the art This may be done by depositing onto a continuous functionalized area on the substrate surface, drops containing the biopolymer or other chemical probes or probe precursors (for example, biomonomers such as nucleoside phosphoramidites) at the multiple feature locations of each array on substrate to be fabricated, so that the probes or probe precursors bind to the linking agent at the feature locations. This step may be repeated at one or more features, particularly when the in situ method of fabricating biopolymers is used, until the arrays are completed. Such procedures are disclosed in detail in, for example, U.S. Pat. Nos. 6,242,266, 6,232,072, 6,180,351, 6,171,797, 6,323,043, U.S. patent application Ser. No. 09/302,898 filed Apr. 30, 1999 by Caren et al., and the references cited therein. Array units of the present invention may be fabricated using any of those apparatus described in the following U.S. Pat. Nos. 6,420,180; 6,323,043; or 6,180,351.

It will be appreciated that an array can be fabricated by other means, such as by photolithographic techniques, as discussed in U.S. Pat. Nos. 6,379,895 and 6,416,952.

In one aspect, a method of the present invention uses a dispensing system to form a liquid drop that covers many individual nucleic acid features. An example of such a dispensing system is discussed in U.S. Pat. No. 6,582,756, assigned to Agilent Technologies, Inc. In one aspect, the size of the drop is adequate to cover a large number of features to achieve a sensible average contact angle for a feature. Also, the liquid dispensed should have a high surface energy, such as water does, so that the dynamic range of the measurement of the contact angle can be extended. For example for propylene carbonate, the surface energy is around 40 dyne/cm and enters a spontaneous wetting regime after only about 20 layers. Water extends the range of the present invention substantially due to its high surface energy (about 72 dyne/cm). The dispensing system may additionally include a camera or other detection system, e.g., such as the FTÅ200, which is a flexible video system (available from First Ten Ångstroms, Portsmouth, Va.) for measuring the contact angle of the drop.

As a result of feature formation, as the polynucleotides are extended at each feature location, each feature of an array becomes less hydrophobic than the functionalized inter-feature or background areas. This is a result of the hydrophilic functional groups present in the extending polynucleotide (although other biopolymers with hydrophilic groups, such as peptides, could be used instead). Each feature is less hydrophobic than inter-feature region on an array, as a result of the presence of the polynucleotides (with their hydrophilic functional groups) at each feature. Similarly, each array on a multi-array substrate is less hydrophobic than an inter-array region. When features are sufficiently closely packed within an array, while the inter-feature areas still retain their unreacted first and second silanes, the overall character of the surface at array will become less hydrophobic than inter-array region.

Regions of greater hydrophobicity have a higher contact angle with an aqueous drop than regions of lower hydrophobicity. Thus, the amount of polynucleotide deposition or the amount of in situ synthesis at features on an array may be monitored by measuring contact angle over features of an array, as a means of assessing the quality of an array. For example, contact angle at a feature should decrease (relative to inter-feature areas) over successive rounds of monomer deposition (e.g., from 1 to about 60) during in situ synthesis to generate polynucleotide probes, eventually becoming constant. In one aspect, features comprising acceptable amounts of synthesis will form a contact angle with a drop of water contacting the features, which ranges from about 0 to about 40 degrees. However, in another aspect, array quality is evaluated by comparing the change in contact angle observed for a test array to the change in contact angle observed for an array determined to have satisfactory quality (e.g., through hybridization experiments). The quality of a multi-array substrate may similarly be evaluated by monitoring contact angles formed by a drop over an array during array synthesis as the contact angle should decrease relative to an inter-array region on a substrate.

In one aspect, determination of array quality in the present invention is based upon determination of an effective contact angle which represents an average of contact angles of drops which include feature and inter-feature regions of an array, where the average is weighted by the surface area occupied by feature and inter-feature regions, respectively. Weighted averages may be determined, for example, using Cassie's equation. See, Cassie, A. B. D. Discuss. Faraday Soc., 3, 11, 1948.Equation (1) shows Cassie's equation as applied to an array of nucleic acids (such as a DNA array), for example.
cos (θ)=ƒ₁ cos (θ₁)+ƒ₂ cos (θ₂). Equation (1)

In Equation (1), f₁ is the fraction of surface area covered by a feature that would make a contact angle of θ₁ with the working liquid if the surface were homogeneously covered with that media. Likewise f₂ and θ₂ are the corresponding surface coverage fraction and contact angles, respectively, corresponding to inter-feature or background regions. θ is the effective contact angle determined by obtaining the weighted average of contact angles θ₁ and θ₂.

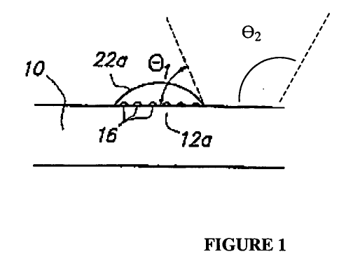

FIG. 1 illustrates a contact angle θ₁ made by a drop 22a contacting features 16 each feature comprising probes, and a contact angle θ₂ made by a drop contacting an inter-feature or background region on a surface 11a of a substrate 10.

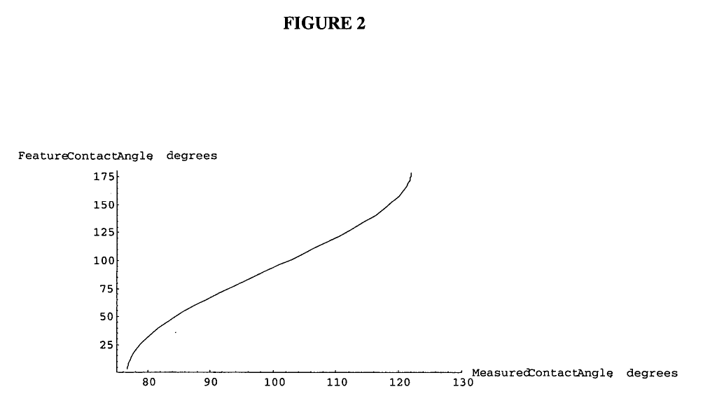

A plot 200 of the calculated feature contact angle versus the measured contact angle is shown in FIG. 2. More particularly, FIG. 2 shows the measured contact angle of a surface patterned with 38% coverage of nucleic acid features and 104-degree background contact angle. It is important to note that there is a lower limit to the measurement of the contact angle where the features have such high surface energy that the measurement of the contact angle becomes saturated. Thus, it is important to use a very high surface energy liquid if possible, e.g., such as water.

If it is assumed that the features are of constant area throughout the process or that at least that the average size of the features at each layer is known, then the contact angle and hence synthesis quality within the feature areas can be estimated. This can be accomplished by simply rearranging Equation 1 to solve for the value for θ as shown in Equation (2).

$$Equation ( 2 ) : \\ θ = {cos}^{- 1} [ \frac{cos ( {θ}_{1} ) - {f}_{2} cos ( {θ}_{2} )}{{f}_{1}} ] .$$

Thus, by knowing the average feature coverage fraction, the background area fraction, the contact angle of the background, and the measured or effective contact angle, a measure of the feature contact angle can be determined.

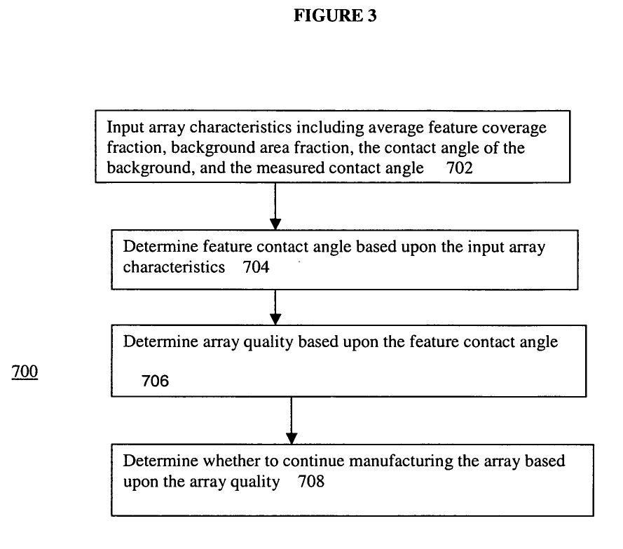

FIG. 3 shows a flowchart 700 for assessing array quality during in-situ manufacturing using contact angle in one embodiment of the invention. As shown in FIG. 3, input 702 characteristics including the average feature coverage fraction, the background area fraction, and the measured contact angle of the background, determine 704 the feature contact angle based upon the input, determine 706 array quality based upon the feature contact angle, and determine 708 whether to continue the manufacturing process for the array based upon the quality.

In one aspect, an array assembly comprises at least one array for assessing polymer mass on the substrate in addition to an array to be contacted with a sample. The “subarray” for assessing polymer mass may comprise fewer features than the array to be contacted with sample. The array assembly may comprise more than one array and/or more than one subarray. In one aspect, the subarray corresponds to a dedicated region for printing patterns of features for contact angle measurement. At each layer of synthesis and/or at suitable intervals, a drop of liquid, such as water, is deposited on the subarray and measured to determine contact angle using techniques discussed above. In one aspect, the array assembly comprises an identifier associated with data relating to feature contact angle for the subarray, and/or data relating to polymer mass on the array correlated to the feature contact angle determined for the subarray. In one aspect, a subarray comprises at least one feature and in general, fewer features than the chemical array for contacting target.

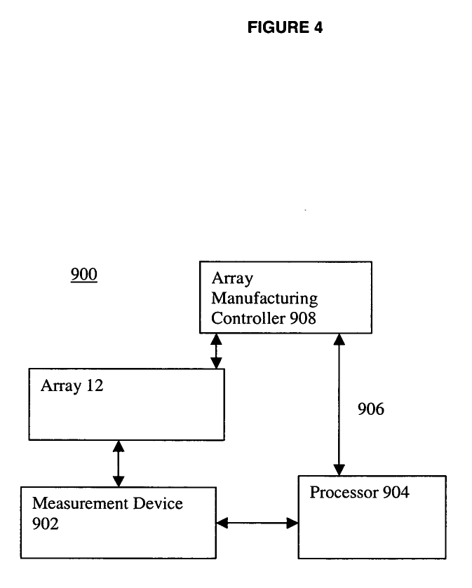

FIG. 4 shows a computer-based system 900 which determines array quality during in-situ manufacturing using contact angle, as in the present invention. In general, a typical computer-based system 900 includes any number of processors (also referred to as central processing units, or CPUs) that can be coupled to one or more storage devices including a random access memory, or RAM, and/or a read only memory, or ROM). A storage device may be used to transfer data and instructions uni-directionally or bi-directionally to the CPU. A storage device may include any suitable computer-readable media, such as a virtual memory, a CD-ROM or DVD-ROM. A computer-based system 900 according to one aspect of the invention may be coupled to an interface that includes one or more input/output devices such as such as video monitors, track balls, mice, keyboards, microphones, touch-sensitive displays, transducer card readers, magnetic or paper tape readers, tablets, styluses, voice or handwriting recognizers, or other well-known input devices such as, of course, other computers. Finally, a CPU optionally may be coupled to a computer or telecommunications network using a network connection as is known in the art. With such a network connection, it is contemplated that the CPU might receive information from the network, or might output information to the network in the course of performing the above-described method steps. The above-described devices and materials will be familiar to those of skill in the computer hardware and software arts.

The hardware elements described above may implement the instructions of multiple software modules for performing the operations of this invention. In addition, embodiments of the present invention further relate to computer readable media or computer program products that include program instructions and/or data (including data structures) for performing various computer-implemented operations. The media and program instructions may be those specially designed and constructed for the purposes of the present invention, or they may be of the kind well known and available to those having skill in the computer software arts. Examples of computer-readable media include, but are not limited to, magnetic media such as hard disks, floppy disks, and magnetic tape; optical media such as CD-ROM, CDRW, DVD-ROM, or DVD-RW disks; magneto-optical media such as optical disks; and hardware devices that are specially configured to store and perform program instructions, such as read-only memory devices (ROM) and random access memory (RAM). Examples of program instructions include both machine code, such as produced by a compiler, and files containing higher level code that may be executed by the computer using an interpreter.

In one aspect, the system further includes a detector for determining one or more of the following properties of the array: average feature coverage fraction, the background area fraction, and the measured contact angle of the background of the array, and the measured contact angle for a drop which covers at least one feature, and in certain aspects, a plurality of features, on the array. In certain aspects, the detector comprises a plurality of detectors. In one aspect, a detector comprises a camera in optical communication with the array, while in another aspect, the detector comprises a video imaging system.

In one aspect, the properties or characteristics of the array are input to a processor 904 of a computer which executes a program stored on computer-readable medium. In one aspect, the program includes instructions for implementing any operations performed in conjunction with any of the methods described above. For example, the program may include instructions for determining an average feature contact angle for a feature on a chemical array. In certain aspects, the program further comprises instructions for comparing an average feature contact angle to a feature contact angle determined for a validated array (e.g., an array for which contact angle has been correlated with an amount of polymer mass at one or more features on an array and/or the target specificity (e.g., ability to specifically bind to a target) of one or more features on the array. In one aspect, the program further comprises instructions to discontinue polymer synthesis on the array when the average feature contact angle differs from a threshold contact angle representing a satisfactory array (e.g., an array comprising an acceptable polymer mass). The processor may output a value 906 relating to the comparison between the average feature contact angle and the threshold contact angle for a validated array, toto an array manufacturing controller 908 indicating whether to continue the manufacturing of the array or abort the manufacturing of the array. In one aspect, the array manufacturing controller communicates with a fluid dispensing system which dispenses polymer at features on the array (e.g., such as an ink-jet printer).

The many features and advantages of the invention are apparent from the detailed specification and, thus, it is intended by the appended claims to cover all such features and advantages of the invention that fall within the true spirit and scope of the invention. Further, since numerous modifications and changes will readily occur to those skilled in the art, it is not desired to limit the invention to the exact construction and operation illustrated and described, and accordingly all suitable modifications and equivalents may be resorted to, falling within the scope of the invention.

What is claimed is:

1. A method for evaluating a chemical array comprising a plurality of features, the method comprising:
determining an average feature coverage fraction on a chemical array;
measuring the contact angle of a drop covering a at least one feature;
determining a background area fraction of the array;
measuring a contact angle of the background;
obtaining an average of the measured contact angles weighted by the amount of surface area covered by features and background.

2. The method of claim 1, further comprising determining an average feature contact angle for the array.

3. The method of claim 1, wherein the average feature contact angle is correlated with polymer mass at a feature.

4. The method of 3, wherein the polymer mass and/or average feature contact angle is compared to the polymer mass and/or average feature angle of a validated array which has been contacted with a sample comprising target molecules.

5. The method of claim 4, wherein binding of target molecules to one or more features of the validated array has been determined.

6. The method of claim 4, wherein the polymer comprises a polynucleotide.

7. The method of claim 6, wherein contact angles are measured after one or more steps of polymer synthesis at features on the array.

8. The method of claim 7, wherein contact angles are measured after each of a plurality of steps of polymer synthesis at features on the array.

9. The method of claim 7, wherein a decision to continue or discontinue polymer synthesis is made based on the determination of contact angles after one or more steps of polymer synthesis.

10. The method of claim 7, wherein a processor determines average feature contact angles after one or more steps of polymer synthesis.

11. The method of claim 10, wherein the processor instructs a polymer deposition system to continue or discontinue polymer deposition on a substrate on which a plurality of polymers is being synthesized at a plurality of locations, thereby forming features on the substrate at the locations.

12. The method of claim 11, wherein a user provides input to the processor regarding whether the deposition system should continue or discontinue polymer deposition.

13. The method of claim 11, wherein the processor automatically instructs the polymer deposition system based on a predetermined threshold for an average feature contact angle.

14. The method of 1, wherein the drop covering at least one feature covers a plurality of features.

15. The method of claim 1, wherein the drop covering at least one feature is formed by a liquid dispensing system.

16. The method of claim 15, wherein the liquid dispensing system comprises an ink jet printer.

17. The method of claim 1, wherein the drop comprises water.

18. The method of claim 1, wherein the drop comprises propylene carbonate.

19. The method of claim 1, wherein the effective contact angle, θ, determined by obtaining the weighted average of a feature contact angle and background contact angle, is determined by the equation:
cos (θ)=ƒ₁ cos (θ₁)+ƒ₂ cos (θ₂), wherein
f₁ is the fraction of surface area covered by a feature that would make a contact angle of θ₁ with a drop of liquid if the surface were homogeneously covered with that liquid,
f₂ is the surface coverage fraction of background regions of the array, and
θ₂ is the contact angles of a drop of liquid contacting a background region.

20. The method of claim 1, wherein contact angle is measured using an imaging system in optical communication with the array.

21. A computer program product comprising a computer readable medium comprising a program for implementing a method of claim 1.

22. The computer program product of claim 21, wherein the program includes instructions for determining an average feature contact angle for a feature on a chemical array.

23. The computer program product of claim 21, wherein the program further comprises instructions for comparing an average feature contact angle to a feature contact angle determined for a validated chemical array.

24. The computer program product of claim 21, wherein the program further comprises instructions for correlating an average feature contact angle to polymer mass at features on the array.

25. A system for performing a method according to claim 10, comprising a processor, a polymer deposition system, and a substrate comprising a chemical array.

26. The system of claim 25, further comprising a detector in optical communication with the array for determining a contact angle of a drop of liquid on the array.

27. The system of claim 25, wherein the processor is connectable to or comprises a memory comprising data relating to average feature contact angles for one or more arrays.

28. The system of claim 27, wherein the data further comprises data relating to one or more properties of the one or more arrays.

29. The system of claim 28, wherein the one or more properties comprise polymer mass at one or more features of the array.

30. The system of claim 28, wherein the one or more properties comprises target specificity of one or more features of the array.

31. The system of claim 25, wherein the array is associated with an identifier.

32. The system of claim 31, wherein the identifier is associated with data relating to average feature contact angle.

33. The system of claim 32, wherein the identifier is associated with data relating to polymer mass on the array.

34. The system of claim 25, wherein the processor receives information relating to the average feature size and number of features on the array and determines the average feature coverage fraction of the array.

35. The system of claim 34, wherein the information is provided by a user.

36. The system of claim 35, wherein the system further comprises a user interface in communication with the processor and the user inputs the information to the user interface.

37. The system of claim 34, wherein information is provided by a detector in optical communication with the array.

38. The system of claim 25, wherein the processor receives information relating to the background coverage fraction.

39. The system of claim 25, wherein processor provides instructions to the polymer deposition system based on an average feature contact angle determined for the array.

40. A substrate comprising:
a chemical array comprising a plurality of addressable features, each feature comprising a probe for binding to a target in a sample;
a subarray for determining an average feature contact angle for features on the chemical array, comprising at least one feature and fewer features than those present in the chemical array.

41. The substrate of claim 40, further associated with an identifier, wherein the identifier is associated with data relating to the average feature coverage fraction and average background coverage fraction of the subarray.
